(19) United States
(12) Patent Application Publication
(10) Pub. No.: US 2010/0114321 A1
(43) Pub. Date: May 6, 2010

(54) HOMUNCULUS

(75) Inventors: Keiichi Akita, Tokyo (JP); Tomoyuki Mochizuki, Tokyo (JP)

Correspondence Address: HOFFMANN & BARON, LLP, 6900 JERICHO TURNPIKE, SYOSSET, NY, 11791, US

(73) Assignee: National University Corporation, Tokyo Medical and Dental University, Tokyo (JP)

(21) Appl. No.: 12/434,149

(22) Filed: May 1, 2009

(30) Foreign Application Priority Data
Nov. 4, 2008 (JP) 2008-283656

(51) Int. Cl. A61F 2/30

(52) U.S. Cl. 623/18.11

(57) ABSTRACT

The object of the present invention is to provide a homunculus of a glenohumeral joint in which the supraspinatus and the infraspinatus are correctly connected to the superior humerus. A homunculus 10 includes a scapula part 1; a superior humerus part 2; a supraspinatus part 3; and an infraspinatus part 4. A tip 32 of the supraspinatus part 3 is fixed to the upper portion of a greater tubercle portion 2a of the superior humerus part 2. One side of a tip 42 of the infraspinatus part 4, the tip extending like a belt, forms a boundary with a tip edge of the supraspinatus part 3. Furthermore, the infraspinatus part 4 is removably engaged with the greater tubercle portion 2a of the superior humerus part 2 such that the tip 42 covers the greater tubercle portion 2a.

This application is based on and claims the benefit of priority from Japanese Patent Application No. 2008-283656, filed on 4 Nov. 2008, the content of which is incorporated herein by reference.

### BACKGROUND OF THE INVENTION

1. Field of the Invention

The present invention relates to a homunculus. Particularly, the present invention relates to a homunculus of a glenohumeral joint with a rotator cuff.

2. Related Art

A homunculus three-dimensionally reproduces a structure of a human body based on anatomical knowledge. The homunculus can serve to train and educate medical personnel. Moreover, the homunculus can be used for explaining treatment to a patient and persons concerned.

As such a homunculus, for example, disclosed is a demonstration model assembly having an anatomical model structure that is stably supported with a support having a comparatively small area on a stable surface (refer to Patent Document 1). The anatomical model structure according to Patent Document 1 is an anatomy of a patient, preferably a reproduction of a vertebral column, and is at least partly and removably embedded to a support.

The glenohumeral joint (scapulohumeral joint) of a human body is anatomically composed of the superior humerus, the clavicle, and the scapula. The rotator cuff is composed of four muscle tendons: the supraspinatus, the infraspinatus, the musculus teres minor, which are attached to the greater tubercle of the superior humerus for the outward rotation of the arm, and the subscapularis that is attached to the lesser tubercle of the superior humerus for the medial rotation of the arm.

[Patent Document 1] Japanese Unexamined Patent Application, First Publication No. 2005-512131

### SUMMARY OF THE INVENTION

Regarding the foot print (connecting aspect) of the rotator cuff, it has been conventionally believed that the supraspinatus is the muscle tendon which is most related to rupture of the rotator cuff. However, atrophy of the infraspinatus has been often observed in patients who have minimum- to intermediate-sized rupture of the rotator cuff. This fact has been poorly explained by the popular understanding of the anatomical connecting structure of the supraspinatus and the infraspinatus. Accordingly, the present inventors have reexamined connecting structure of the brachium (humeral insertion) regarding the tendons of the supraspinatus and the infraspinatus.

As a result of investigating shoulders of a number of specimens, the foot print of the supraspinatus in the greater tubercle of the humerus is found to be considerably smaller than what was conventionally believed. Moreover, this region of the greater tubercle of the humerus is practically occupied by a substantial amount of the infraspinatus.

In other words, this fact shows that rupture of the rotator cuff, which, according to conventional theory is primarily caused by the tendon of the supraspinatus, also includes considerable influence from the infraspinatus.

The rupture of the rotator cuff often occurs to aged patients as a result of degenerative changes due to aging or as a result of injury. The rupture of the rotator cuff then leads to dysfunction of the shoulder. Therefore, it is important to identify the correct location and range of the rupture in order to facilitate an appropriate surgical treatment for functional recovery.

The rupture is analyzed using echography and MRI before surgery, and is diagnosed based on surgical findings. In both examinations, imaging of the bone surfaces of the greater tubercle and the lesser tubercle provides important findings in many cases in order to investigate the damaged tendons.

Most anatomical textbooks describe that the supraspinatus is connected to the uppermost position of the greater tubercle of the humerus, and the infraspinatus is connected to the intermediate position of the greater tubercle of the humerus.

Most commercial models of a glenohumeral joint with a rotator cuff have a structure, in which most of the supraspinatus is connected to the greater tubercle of the humerus, and a little of the infraspinatus is connected to the greater tubercle of the humerus. Since the models of a glenohumeral joint with such a structure do not reflect the correct structure of the rotator cuff, it is difficult to perform surgical treatment for rupture of the rotator cuff. Moreover, it is difficult to correctly understand the structure of the rotator cuff.

If a model of a glenohumeral joint with correctly structured rotator cuff can be actualized, it can serve to train and educate medical personnel. If a model of a glenohumeral joint with a correctly structured rotator cuff can be actualized, it is also possible to facilitate the correct understanding by patients and persons concerned of the treatment for the rupture.

The present invention has been made in view of such problems, and the object of the present invention is to provide a homunculus of a glenohumeral joint with a rotator cuff, in which at least the supraspinatus and the infraspinatus are correctly connected to the greater tubercle of the superior humerus.

The present inventors have completed the present invention by finding that: the supraspinatus originates from the supraspinous fossa of the scapula and the inner face of the supraspinatus fascia, runs outward under the acromial, and reaches and is connected to the upper portion of the greater tubercle of the humerus; the infraspinatus originates from the infraspinous fossa of the scapula and a wide range of an inner face of the supraspinatus fascia, and the muscle bundle converges outward and reaches the anterior portion of the greater tubercle of the humerus; and the end portion of this infraspinatus is elastically connected to the greater tubercle of the humerus. More specifically, the present invention provides the following.

According to a first aspect of the present invention, provided is a homunculus of a glenohumeral joint of a human body, the homunculus including: a scapula part that imitates the scapula; a superior humerus part that imitates the superior humerus; a supraspinatus part that imitates the supraspinatus; and an infraspinatus part that imitates the infraspinatus, in which an end of the supraspinatus part is fixed to a supraspinous fossa portion of the scapula part, and a tip of the supraspinatus part is fixed to an upper portion of a greater tubercle portion of the superior humerus part, and in which, an end of the infraspinatus part is fixed to an infraspinous fossa portion of the scapula part, one side of a tip of the infraspinatus part, the tip extending like a belt, forms a boundary with a tip edge of the supraspinatus part, and the tip is engaged with the greater tubercle portion of the superior humerus part such that the tip covers the greater tubercle portion.

The homunculus according to the invention of the first aspect is a homunculus that imitates the glenohumeral joint of a human body, and is provided with a scapula part and a superior humerus part. The scapula part imitates the scapula. The superior humerus part imitates the superior humerus.

Moreover, the homunculus according to the invention of the first aspect is provided with a supraspinatus part and an infraspinatus part. The supraspinatus part imitates the supraspinatus. The infraspinatus part imitates the infraspinatus.

An end of the supraspinatus part is fixed to the supraspinous fossa portion of the scapula part. Moreover, a tip of the supraspinatus part is fixed to the upper portion of a greater tubercle portion of the superior humerus part.

An end of the infraspinatus part is fixed to the infraspinous fossa portion of the scapula part. Moreover, one side of a tip of the infraspinatus part, the tip extending like a belt, forms a boundary with a tip edge of the supraspinatus part. Furthermore, the infraspinatus part is engaged with the greater tubercle portion of the superior humerus part such that the tip of the infraspinatus part covers the greater tubercle portion.

The glenohumeral joint of a human body is anatomically composed of the superior humerus, the clavicle, and the scapula. Therefore, the homunculus according to the first aspect of the invention does not necessarily exclude providing a clavicle part that imitates the clavicle. Moreover, the homunculus according to the first aspect of the invention may be a model of the right glenohumeral joint, may be a model of the left glenohumeral joint, or may be a model including the right and left glenohumeral joints.

Here, the scapula part imitating the scapula may be a scapula part imitating shapes and positions of each site which are characteristic of the scapula of a human body, or may not necessarily be substantially the same replica, or may be a model that is reduced in size as a similar figure. Threaded holes, fitting holes, steps or protrusions may be provided to the scapula part in order to attach other imitation parts.

Similarly, the superior humerus part imitating the superior humerus may be a superior humerus part imitating shapes and positions of each site which are characteristic of the superior humerus of a human body, or may not necessarily be substantially the same replica, or may be an analogous model that is reduced in size. Threaded holes, fitting holes, steps or protrusions may be provided to the superior humerus part in order to attach other imitation parts.

The scapula part and the superior humerus part may be manufactured from timber, or may be manufactured by curing gypsum, and the material is not limited as long as the product is solid. It is preferable to mold a synthetic resin for mass production, thereby making it possible to obtain scapula parts and superior humerus parts with desirable shapes. Hard chemical rubber is also included in the synthetic resin.

The supraspinatus and the infraspinatus are classified as skeletal muscles, and are also classified as voluntary muscles which can be consciously moved. The supraspinatus part and the supraspinatus part imitate the skeletal muscles consisting of soft tissues, and therefore are preferably made of soft chemical rubber.

In anatomy, connective tissues, which are in a portion where the skeletal muscles are attached to a bone, and which are toward a main portion of the skeletal muscles, are defined as “tendon”, and are present as being attached to the skeletal muscles of a human body. Since it is supposed that the skeletal muscles exhibit a red color as a whole and the tendons exhibit a white color, it is preferable for the supraspinatus part and the infraspinatus part to be colored red, and the tendon parts to be colored white.

However, it is more preferable for the scapula part and the superior humerus part to be colored white, and the supraspinatus part and the infraspinatus part to be colored red as a whole, since the muscles and the bones can be easily distinguished. The end and the tip of the supraspinatus part or the infraspinatus part are fixed or engaged with the scapula part or the superior humerus part, thereby making it possible to realize that these are portions of the tendons.

The fixing of the end and the tip of the supraspinatus part to the scapula part and the superior humerus part does not mean fixing by which separation is difficult. It is preferable to removably fix the supraspinatus part with fastenings such as screws. Moreover, it is preferable to removably fix the supraspinatus part with a face fastener as described later.

Similarly, fixing of the end of the infraspinatus part to the scapula part does not mean fixing by which separation is difficult. It is preferable to removably fix the infraspinatus part with fastenings such as screws. Moreover, it is preferable to removably fix the infraspinatus part with a face fastener as described later.

On the other hand, for example, as a manner of removably engaging the infraspinatus part with the greater tubercle portion, a protrusion may be provided to the greater tubercle portion, an engaging hole may be provided to the tip of the infraspinatus part, and the engaging hole may be engaged with the protrusion. It is preferable to pull and engage the tip of the infraspinatus part.

The homunculus according to the first aspect of the invention imitates the attachment positions, which have been discovered by the present inventors, of the supraspinatus and the infraspinatus in a shoulder of a human body. One side of the tip of the infraspinatus part, the tip extending like a belt, forms a boundary with the tip edge of the supraspinatus part, and the tip is engaged with the greater tubercle portion of the superior humerus part such that the tip covers the greater tubercle portion. Accordingly, it is possible to understand that the infraspinatus acts on the rotation of the shoulder to a greater extent than the supraspinatus does. In other words, it is possible to understand that the infraspinatus is the muscle tendon which is most related to rupture of the rotator cuff.

The homunculus according to the first aspect of the invention actualizes the model of the glenohumeral joint with the rotator cuff of the correct structure with regards to the attachment positioning of the supraspinatus and the infraspinatus in the shoulder of the human body, and therefore can serve to train and educate medical personnel. Moreover, the homunculus according to the first aspect of the invention also enables a patient and persons concerned to understand the treatment for a rupture.

According to a second aspect of the present invention, the homunculus as recited in the first aspect is provided, in which one side of the tip of the infraspinatus part is removably engaged with a high position of the greater tubercle portion, and an other side of the tip of the infraspinatus part is removably engaged with an intermediate position of the greater tubercle portion.

According to a third aspect of the present invention, the homunculus as recited in the first or second aspect is provided, in which the supraspinatus part and the infraspinatus part are formed from an elastic member.

According to a fourth aspect of the present invention, the homunculus as recited in the second or third aspect is provided, in which the infraspinatus part has: a first surface fastener that removably fixes an end of the infraspinatus part to the scapula part; and a second surface fastener that removably fixes a tip of the infraspinatus part to the superior humerus part.

According to a fifth aspect of the present invention, the homunculus as recited in any one of the first to fourth aspects is provided, the homunculus further including: a base that fixes at least one of the scapula part and the superior humerus part; a cover that covers the base; and a transparent cover, which is partly provided to the cover, and through which a joint portion is visible, in which the transparent cover has a plurality of apertures through which an endoscope or a surgical appliance can be inserted.

In the homunculus according to the fifth aspect of the invention, an endoscope or a surgical appliance can be inserted into the plurality of apertures, thereby making it possible to perform training of an endoscopic operation (surgery in which a hole is opened with a scalpel, observation is performed with a camera, and treatment is performed). As compared to a surgical operation, the endoscopic operation is supposed to be advantageous in that injuries of tissues such as normal skin or muscles are fewer, scars are less conspicuous, less pain is suffered, movement is less limited, etc. In this case, by using a retractable cloth body as the infraspinatus part, it is possible to preferably perform training of an endoscopic operation for a rupture.

In the homunculus according to the present invention, one side of the tip of the infraspinatus part, the tip extending like a belt, forms a boundary with the tip edge of the supraspinatus part, and the tip is engaged with the greater tubercle portion of the superior humerus part such that the tip covers the greater tubercle portion. Accordingly, it is possible to understand that the infraspinatus acts on the rotation of the shoulder to a greater degree than the supraspinatus does. The homunculus according to the present invention makes it possible to understand that the infraspinatus is the muscle tendon which is most related to rupture of the rotator cuff.

The homunculus according to the present invention actualizes the model of the glenohumeral joint with the rotator cuff with the correct structure with regard to the attachment positioning of the supraspinatus and the infraspinatus in a shoulder of a human body, and therefore can serve to train and educate medical personnel. Moreover, the homunculus according to the present invention also enables a patient and persons concerned to understand the treatment for a rupture.

### BRIEF DESCRIPTION OF THE DRAWINGS

FIG. 1 is a captured image of the top face of the right shoulder;

FIG. 2 is a captured image of the top face of the right shoulder;

FIG. 3 is a captured image of the top face of the right shoulder;

FIG. 4 is a captured image of the top face of the right shoulder;

FIG. 5 is a front view of the scapula in the right shoulder;

FIG. 6 is a rear view of the scapula in the right shoulder;

FIG. 7 is a left face view of the scapula in the right shoulder;

FIG. 8 is a front view of the humerus constituting the right arm, and an illustration of the lower portion of the humerus is omitted;

FIG. 9 is a rear view of the humerus constituting the right arm, and an illustration of the lower portion of the humerus is omitted;

FIG. 10 is a perspective view of a homunculus according to one embodiment of the present invention, and the homunculus is viewed from the dorsal side;

FIG. 11 is a front view of the homunculus according to the embodiment;

FIG. 12 is a rear view of the homunculus according to the embodiment;

FIG. 13 is a right-side view of the homunculus according to the embodiment; and

FIG. 14 is a perspective view of a homunculus according to another embodiment of the present invention, and the homunculus according to the another embodiment is viewed from the dorsal side.

### DETAILED DESCRIPTION OF THE INVENTION

The mode for carrying out the present invention is hereinafter described with reference to the drawings.

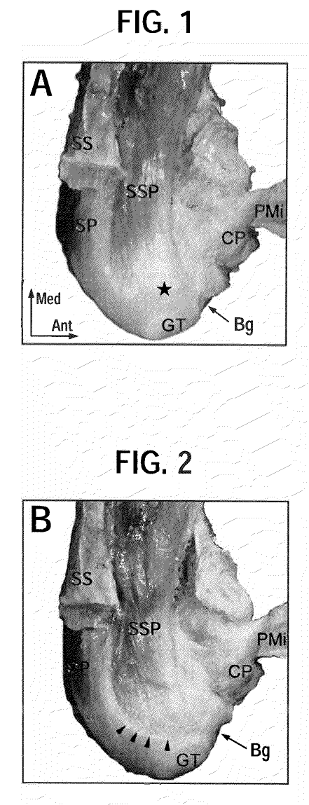

FIG. 1 is a captured image of the top face of the right shoulder;

FIG. 2 is a captured image of the top face of the right shoulder;

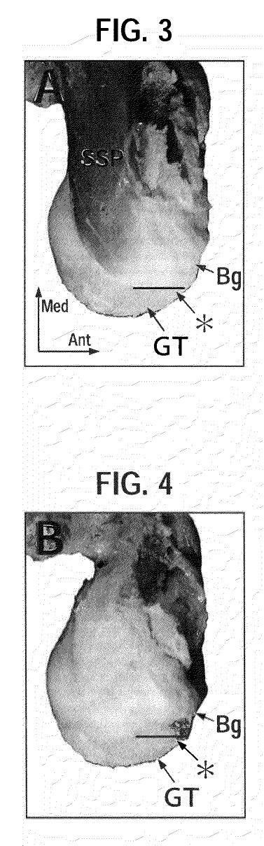

FIG. 3 is a captured image of the top face of the right shoulder;

FIG. 4 is a captured image of the top face of the right shoulder;

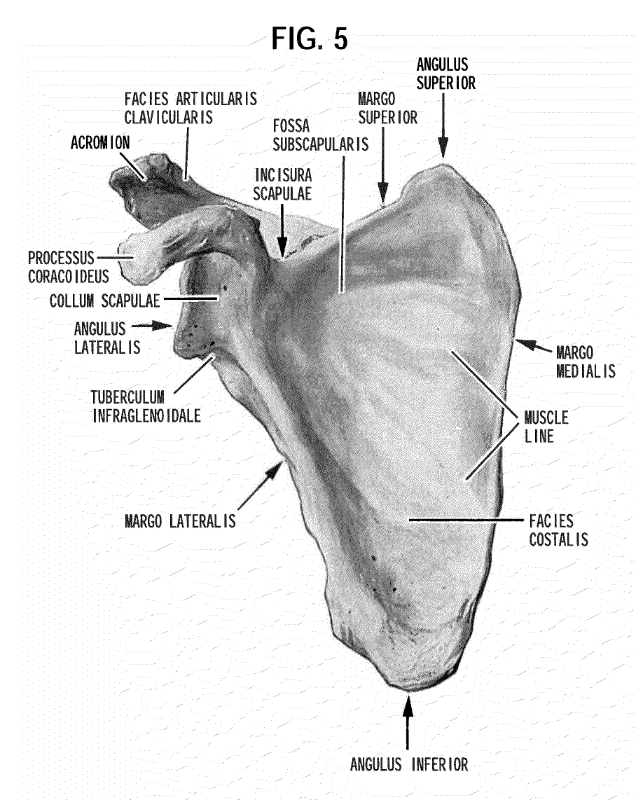

FIG. 5 is a front view of the scapula in the right shoulder;

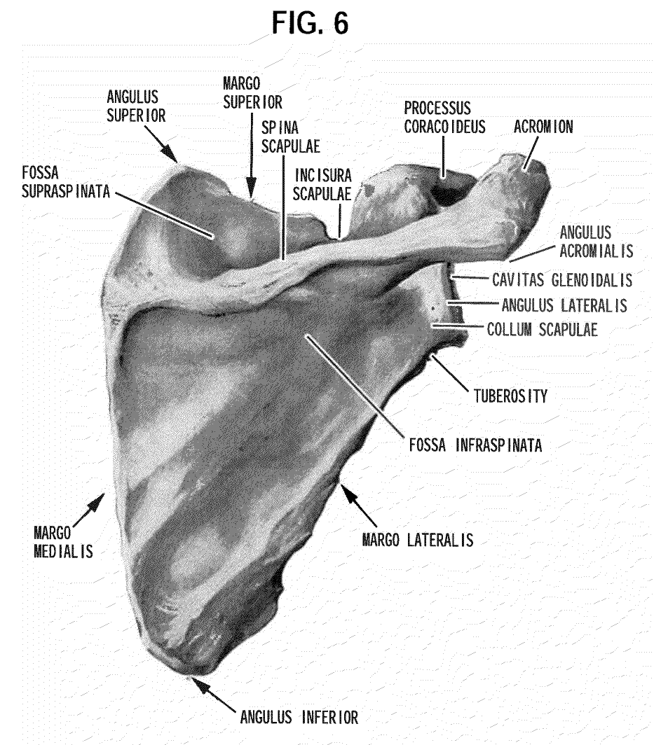

FIG. 6 is a rear view of the scapula in the right shoulder;

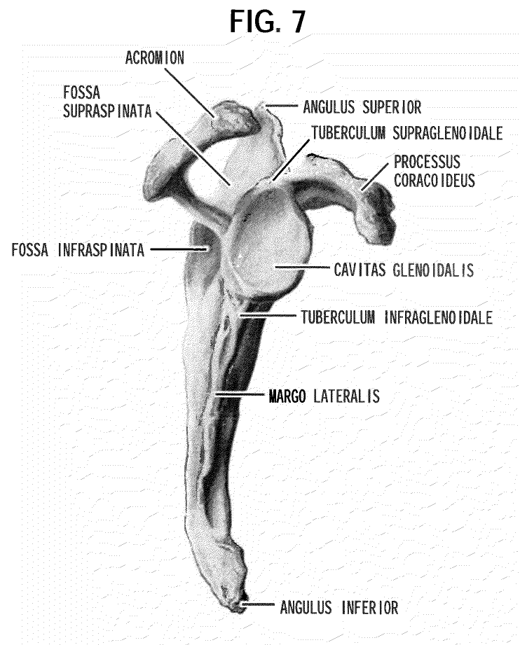

FIG. 7 is a left face view of the scapula in the right shoulder;

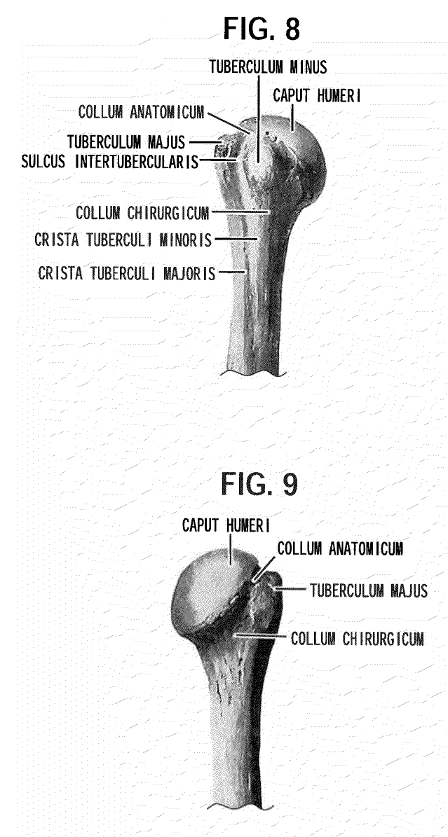

FIG. 8 is a front view of the humerus constituting the right arm, and an illustration of the lower portion of the humerus is omitted;

FIG. 9 is a rear view of the humerus constituting the right arm, and an illustration of the lower portion of the humerus is omitted;

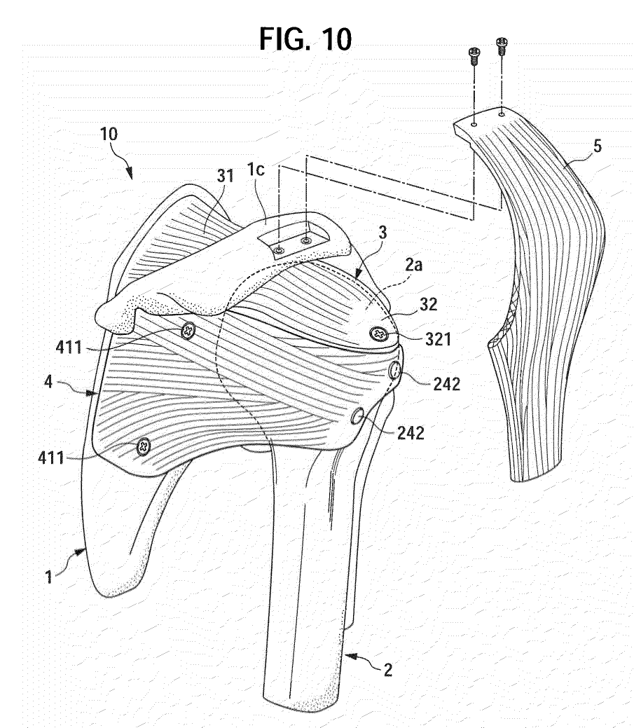

FIG. 10 is a perspective view of a homunculus according to one embodiment of the present invention, and the homunculus is viewed from the dorsal side;

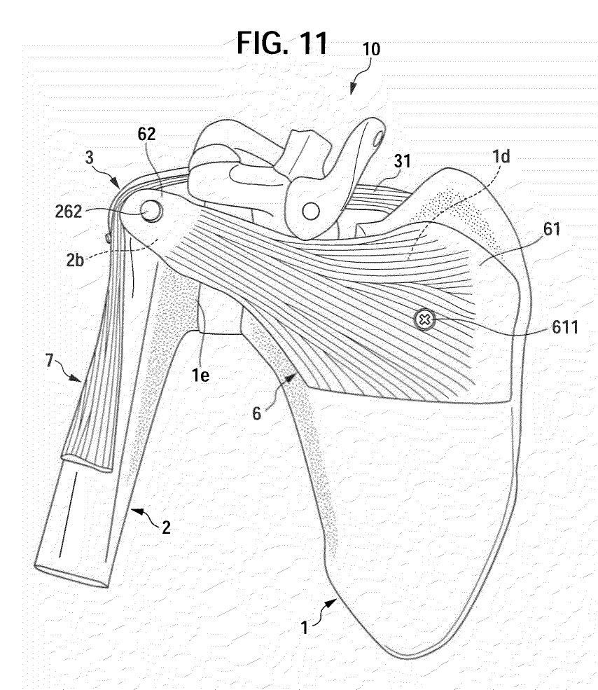

FIG. 11 is a front view of the homunculus according to the embodiment;

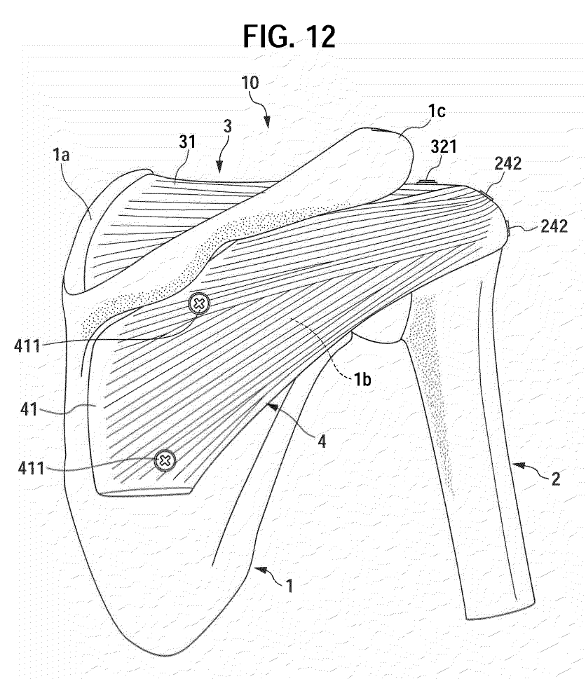

FIG. 12 is a rear view of the homunculus according to the embodiment;

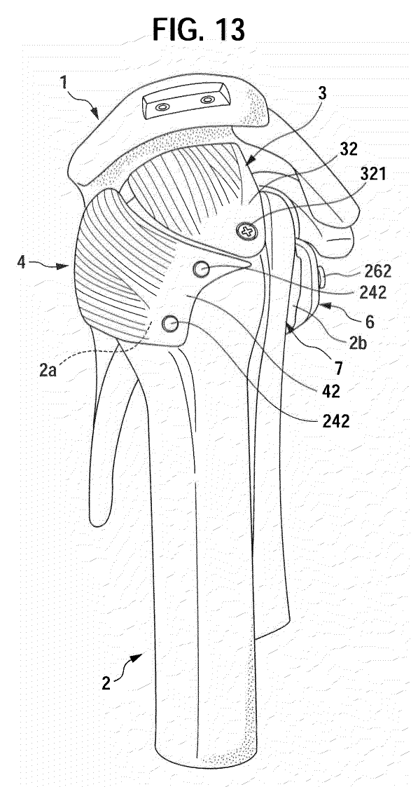

FIG. 13 is a right-side view of the homunculus according to the embodiment; and

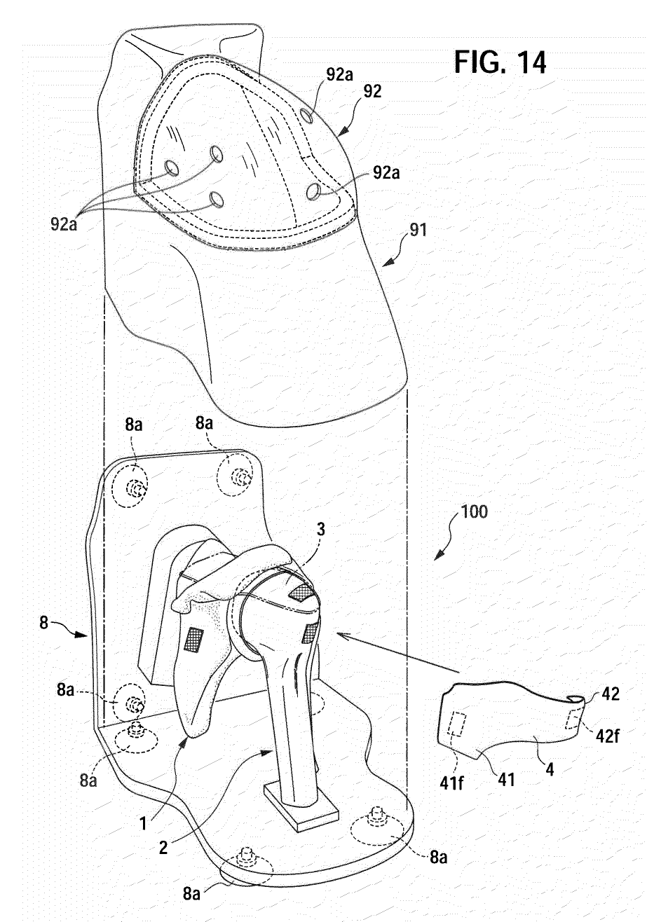

FIG. 14 is a perspective view of a homunculus according to another embodiment of the present invention, and the homunculus according to the another embodiment is viewed from the dorsal side.

First, the structure of the rotator cuff based on the latest discovery according to the present inventors is described. FIGS. 1 and 2, in which the acromion is exfoliated, show tissues of muscles and tendons in the supraspinatus and the infraspinatus which adhere to the greater tubercle. With reference to FIG. 1, the connective tissues cover the supraspinatus SSP and the infraspinatus ISP in the vicinity of the accretion indicated by a star. The connective tissues are manufactured so as to make it difficult to separate the supraspinatus SSP from the infraspinatus ISP.

With reference to FIG. 2, the anterior margin of the supraspinatus SSP indicated by an arrow in the drawing has been clearly confirmed in a portion after removing the connective tissues. It should be noted that, in FIGS. 1 and 2, Bg denotes bicipital groove, CP denotes coracoid process, GT denotes greater tubercle, PMi denotes pectoralis minor, and SS denotes spina scapulae, respectively. Moreover, in FIG. 1 and FIG. 2, the direction of an orthogonal coordinate axis Ant indicates the anticus of the human body, and the direction of an orthogonal coordinate axis Med indicates the median of the human body.

FIGS. 3 and 4 show the tendon accretions in the supraspinatus and the infraspinatus. With reference to FIG. 3, the supraspinatus SSP remains intact, while most of the infraspinatus is exfoliated from the humerus. The line segment indicated by an asterisk shows a boundary of the adhesion region of the supraspinatus and the infraspinatus.

With reference to FIG. 4, the supraspinatus is exfoliated from the humerus. Moreover, the adhesion region of the supraspinatus is observable. In addition, it is of interest that the articular capsule is intact after removing the supraspinatus and the infraspinatus. It should be noted that, in FIGS. 3 and 4, Bg denotes bicipital groove, and GT denotes greater tubercle, respectively.

Next, the findings by the present inventors are described with additional reference to the scapula and the superior humerus shown in FIGS. 5 to 9. In FIGS. 1 to 4, the accretions of the supraspinatus and the infraspinatus to the humerus have the same pattern in all the specimens regarding the anatomy of the infraspinatus and the supraspinatus.

With reference to FIGS. 5 to 7, the supraspinatus originates from the supraspinous fossa and the top surface of the spina scapulae, and reaches the outside. The infraspinatus originates from both the infraspinous fossa and the lower surface of the spina scapulae, and reaches the upper outside.

It has been believed that the accretions of the supraspinatus and the infraspinatus are joined as one structure to the humerus. However, in all the specimens, the ligamentum coracohumerale and the residue of the moderate connective tissues covered the supraspinatus and the infraspinatus in the vicinity of the accretion of the infraspinatus in FIGS. 1 and 2. The anterior margin of the infraspinatus formed an edge, and the boundary between the two muscles was clear (refer to FIGS. 1 and 2).

In FIGS. 1 and 2, the anterior margin of the infraspinatus slightly bulges from the posterior part thereof, and is adjacent to the edge of the supraspinatus. The anterior portion of the infraspinatus partly covers the posterior side of the supraspinatus (refer to FIG. 2).

Next, the infraspinatus was intactly detached from the scapula and the humerus. In this process, the articular capsule between the supraspinatus and the infraspinatus was not damaged. It is said that the top surface of the greater tubercle is characterized by three (high, medium and low) arrest sites. The present inventors discovered that the accretions of the infraspinatus to the humerus sealed about a half of the high arrest site and the entirety of the medium arrest site. In FIG. 3, the foremost region of the accretions of the infraspinatus to the humerus reached the anterior margin of the high arrest site of the greater tubercle.

Next, the present inventors exfoliated the supraspinatus from the scapula and the humerus. It was possible to separate the supraspinatus from the lying articular capsule that was ensured to remain intact during this process. It was discovered that this supraspinatus was inserted into the anteromedial portion of the high arrest site of the greater tubercle.

The foot print of the supraspinatus had a shape of an equilateral triangle with a base lying along the joint surface as one side of the triangle. The foot print of the supraspinatus was anteriorly enlarged, and was posteriorly narrowed (refer to FIG. 4). In the greater tubercle, the foot print of the supraspinatus and the infraspinatus showed the same pattern in all the specimens.

Next, a structure of the homunculus according to the embodiment of the present invention is described. A homunculus 10 shown in FIGS. 10 to 13 imitates the right glenohumeral joint of a human body. With reference to FIGS. 10 to 13, the homunculus 10 is provided with a scapula part 1 and a superior humerus part 2. The scapula part 1 imitates the scapula. The superior humerus part 2 imitates the superior humerus that is pivotally connected to the scapula.

Moreover, with reference to FIGS. 10 to 13, the homunculus 10 is provided with a supraspinatus part 3 and an infraspinatus part 4. The supraspinatus part 3 imitates the supraspinatus. The supraspinatus originates from the supraspinous fossa of the scapula and the inner face of the supraspinatus fascia, runs outward under the acromial, and reaches the upper portion of the greater tubercle of the superior humerus.

With reference to FIGS. 10 to 13, an end 31 of the supraspinatus part 3 is fixed to a supraspinous fossa portion 1a of the scapula part 1. Moreover, a tip 32 of the supraspinatus part 3 is fixed to the upper portion of a greater tubercle portion 2a of the superior humerus part 2.

With reference to FIGS. 10 to 13, an end 41 of the infraspinatus part 4 is fixed to an infraspinous fossa portion 1b of the scapula part 1. Moreover, one side of a tip 42 of the infraspinatus part 4, the tip extending like a belt, forms a boundary with a tip edge of the supraspinatus part 3. Furthermore, the infraspinatus part 4 is removably engaged with the greater tubercle portion 2a of the superior humerus part 2 such that the tip 42 covers the greater tubercle portion 2a.

With reference to FIG. 10, the homunculus 10 is provided with a musculus deltoideus part 5 that imitates the musculus deltoideus. An end of an acromion portion of the musculus deltoideus part 5 is fixed to an acromion portion 1c of the scapula part 1. A tip of the musculus deltoideus part 5 is terminated at acromion portion of the musculus deltoideus part 5. The acromion portion of the musculus deltoideus shoulder can abduct the brachium. It should be noted that an illustration of the musculus deltoideus part 5 is omitted in FIGS. 11 to 13.

With reference to FIG. 11, the homunculus 10 is provided with a subscapularis part 6 that imitates the subscapularis. An end 61 of the subscapularis part 6 is fixed to a subscapular fossa portion 1d of the scapula part 1. The muscle bundles of the subscapularis part 6 gather outward as a triangular shape, and a tip 62 of the subscapularis part 6 is removably engaged with a lesser tubercle portion 2b of the superior humerus part 2. The subscapularis enables medial rotation and horizontal bending of the glenohumeral joint.

With reference to FIG. 11 or 13, the homunculus 10 is provided with a musculus teres minor part 7 that imitates the musculus teres minor. An end of the musculus teres minor part 7 is fixed to the posterior surface lateral margin upper portion 1e of the scapula part 1, and the musculus teres minor part 7 passes between the greater tubercle portion 2a and the lesser tubercle portion 2b of the superior humerus part 2. Although the musculus teres minor terminates at the bottom of the greater tubercle, the illustrated musculus teres minor part 7 is not fixed. The musculus teres minor can outwardly rotate or adduct the glenohumeral joint.

In FIGS. 10 to 13, the supraspinatus part 3, the infraspinatus part 4, the subscapularis part 6 and the musculus teres minor part 7 constitute the rotator cuff that pivots the superior humerus part 2 in relation to the scapula part 1. In other words, the homunculus 10 is a homunculus of the glenohumeral joint with the rotator cuff.

Next, operations of the homunculus according to the embodiment of the present invention are described while complementing the structure.

With reference to FIGS. 10 to 12, the end 31 of the supraspinatus part 3 is fixed to the supraspinous fossa portion 1a of the scapula part 1 by way of a screw (not shown). Moreover, the tip 32 of the supraspinatus part 3 is fixed to the upper portion of the greater tubercle portion 2a of the superior humerus part 2 by way of a screw 321.

In FIGS. 10 to 12, the supraspinatus part 3 is formed from a soft synthetic resin. By moving the superior humerus part 2, the supraspinatus part 3 expands and contracts, thereby making it possible to understand that the supraspinatus is acting on the abduction of the brachium (a membrum is moved away from the central plane of the body).

With reference to FIGS. 10 to 12, the end 41 of the infraspinatus part 4 is fixed to the infraspinous fossa portion 1b of the scapula part 1 by way of two screws 411. Moreover, one side of the tip 42 of the infraspinatus part 4, the tip extending like a belt, forms the boundary with the tip edge of the supraspinatus part 3 (refer to FIG. 13).

With reference to FIG. 13, the tip 42 of the infraspinatus part 4 is removably engaged with two hooks 242 protruding from the greater tubercle portion 2a of the superior humerus part 2 such that the tip 42 covers the greater tubercle portion 2a. As for the tip 42 of the infraspinatus part 4, one side thereof is removably engaged with the hook 242 in the high position of the greater tubercle portion 2a, and the other side thereof is removably engaged with the hook 242 in the medium position of the greater tubercle portion 2a. The infraspinatus part 4 is formed from a soft synthetic resin. By moving the superior humerus part 2, the infraspinatus part 4 expands and contracts, thereby making it possible to understand that the infraspinatus is acting on the outward rotation of the brachium (in a case of the glenohumeral joint, the antebrachium extending anteriorly with the elbow bending, moves inwards).

With reference to FIG. 11, an end 61 of the subscapularis part 6 is fixed to the subscapular fossa portion 1d of the scapula part 1 by way of a screw 611. On the other hand, the tip 62 of the subscapularis part 6 is removably engaged with a hook 262 protruding from the lesser tubercle portion 2b of the superior humerus part 2.

With reference to FIG. 11, the subscapularis part 6 is formed from a soft synthetic resin. By moving the superior humerus part 2, the subscapularis part 6 expands and contracts, thereby making it possible to understand that the subscapularis is acting on the medial rotation of the brachium (in a case of the glenohumeral joint, the antebrachium extending anteriorly with the elbow bending, moves outwards) as well as the horizontal bending thereof.

As for the conventional commercial homunculus of the glenohumeral joint with the rotator cuff, the tip of the supraspinatus part has been fixed so as to cover the greater tubercle portion 2a of the superior humerus part 2 (refer to FIG. 13). Moreover, as for the conventional homunculus, the tip of the infraspinatus part has been engaged with the posterior of the greater tubercle portion 2a of the superior humerus part 2 by way of a hook (refer to FIG. 13). The reason is that it has been understood that most of the tendons of the supraspinatus are connected to the greater tubercle portion of the superior humerus. Accordingly, it has been understood that the rupture of the rotator cuff is caused by injury to the supraspinatus.

On the other hand, with reference to FIG. 13, as for the homunculus 10 according to the embodiment of the present invention, the tip 32 of the supraspinatus part 3 is fixed to the upper portion of the greater tubercle portion 2a of the superior humerus part 2. Moreover, one side of a tip 42 of the infraspinatus part 4, the tip extending like a belt, forms a boundary with a tip edge of the supraspinatus part 3. Furthermore, the tip 42 of the infraspinatus part 4 is removably engaged with the hooks 242 protruding from the greater tubercle portion 2a of the superior humerus part 2 such that the tip 42 covers the greater tubercle portion 2a.

The homunculus 10 according to the embodiment of the present invention is manufactured based on the discovery of the present inventors. In the case of the homunculus 10, it is easy to understand that most of the tendons of the infraspinatus are connected to the greater tubercle portion of the superior humerus. Moreover, it is possible to correctly understand that the rupture of the rotator cuff is caused by injury to the infraspinatus.

In this way, the homunculus 10 according to the embodiment of the present invention actualizes the model of the glenohumeral joint with the rotator cuff with the correct structure with regards to the attachment positioning of the supraspinatus and the infraspinatus in the shoulder of a human body, and therefore can serve to train and educate medical personnel. Moreover, the homunculus 10 according to the embodiment of the present invention also enables a patient and the persons concerned to understand the treatment for rupture.

Next, a structure of the homunculus according to another embodiment of the present invention is described. It should be noted that, in the another embodiment, since components with reference numerals that are the same as the reference numerals used in the preceding embodiment are assumed to have the same operations, descriptions thereof may be omitted.

With reference to FIG. 14, a homunculus 100 according to the another embodiment imitates the right glenohumeral joint of a human body. The homunculus 100 is provided with the scapula part 1 and the superior humerus part 2. Moreover, the homunculus 100 is provided with the supraspinatus part 3 and the infraspinatus part 4.

With reference to FIG. 14, a first surface fastener 41f and a second surface fastener 42f are attached to one face of the infraspinatus part 4. The first surface fastener 41f can removably fix the end 41 of the infraspinatus part 4 to the scapula part 1. The second surface fastener 42f can removably fix the tip of the infraspinatus part 4 to the superior humerus part 2.

With reference to FIG. 14, the homunculus 100 is provided with an L-shaped base 8 and a cover 91 that covers the base 8. The base 8 removably fixes an end face of the scapula part 1 as well as an end face of the superior humerus part 2 by way of fastenings (not shown) such as screws.

With reference to FIG. 14, the homunculus 100 is further provided with a transparent cover 92, which is partly provided to the cover 91, and through which the joint portion of the homunculus 100 is visible. The transparent cover 92 opens a plurality of apertures 92a, through which an endoscope or a surgical appliance (none of which is shown) can be inserted.

Next, operations of the homunculus according to the another embodiment of the present invention are described.

With reference to FIG. 14, a plurality of suckers 8a are dispersedly arranged on the horizontal table and the vertical table of the base 8. Accordingly, it is possible to fix the homunculus 100 on a desk or a wall.

Moreover, with reference to FIG. 14, by use of the homunculus 100, medical personnel can perform training of an endoscopic operation by inserting an endoscope or a surgical appliance in any of the apertures constituting the plurality of apertures. For example, by manufacturing the infraspinatus part 4 with a retractable cloth body, it is possible to perform training of an endoscopic operation for a rupture, in which the infraspinatus part 4 is pulled from the end 41 toward the tip 42.

In the homunculus 100 according to the another embodiment of the present invention, one side of the tip 42 of the infraspinatus part 4, the tip extending like a belt, forms a boundary with the tip edge of the supraspinatus part 3, and the tip 42 of the infraspinatus part 4 is removably engaged with the greater tubercle portion 2a of the superior humerus part 2 such that the tip 42 covers the greater tubercle portion 2a. Accordingly, it is possible to understand that the infraspinatus acts on the rotation of the shoulder to a greater extent than the supraspinatus does. In addition, it is possible to perform training of an endoscopic operation for a rupture attributed to the infraspinatus.

What is claimed is:

1. A homunculus of a glenohumeral joint of a human body, the homunculus comprising:
a scapula part that imitates a scapula;
a superior humerus part that imitates a superior humerus;
a supraspinatus part that imitates a supraspinatus; and
an infraspinatus part that imitates an infraspinatus,
wherein an end of the supraspinatus part is fixed to a supraspinous fossa portion of the scapula part, and a tip of the supraspinatus part is fixed to an upper portion of a greater tubercle portion of the superior humerus part, and
wherein, an end of the infraspinatus part is fixed to an infraspinous fossa portion of the scapula part, one side of a tip of the infraspinatus part, the tip extending like a belt, forms a boundary with a tip edge of the supraspinatus part, and the tip is engaged with the greater tubercle portion of the superior humerus part such that the tip covers the greater tubercle portion.

2. The homunculus according to claim 1, wherein one side of the tip of the infraspinatus part is removably engaged with a high position of the greater tubercle portion, and an other side of the tip of the infraspinatus part is removably engaged with an intermediate position of the greater tubercle portion.

3. The homunculus according to claim 2, wherein the supraspinatus part and the infraspinatus part are formed from an elastic member.

4. The homunculus according to claim 1, wherein the supraspinatus part and the infraspinatus part are formed from an elastic member.

5. The homunculus according to claim 4, wherein the infraspinatus part has: a first surface fastener that removably fixes an end of the infraspinatus part to the scapula part; and a second surface fastener that removably fixes a tip of the infraspinatus part to the superior humerus part.

6. The homunculus according to claim 3, wherein the infraspinatus part has: a first surface fastener that removably fixes an end of the infraspinatus part to the scapula part; and a second surface fastener that removably fixes a tip of the infraspinatus part to the superior humerus part.

7. The homunculus according to claim 2, wherein the infraspinatus part has: a first surface fastener that removably fixes an end of the infraspinatus part to the scapula part; and a second surface fastener that removably fixes a tip of the infraspinatus part to the superior humerus part.

8. The homunculus according to claim 1, further comprising:
a base that fixes at least one of the scapula part and the superior humerus part;
a cover that covers the base; and
a transparent cover, which is partly provided to the cover, and through which a joint portion is visible,
wherein the transparent cover has a plurality of apertures through which an endoscope or a surgical appliance can be inserted.
